(19) United States
(12) Patent Application Publication
(10) Pub. No.: US 2022/0062516 A1
(43) Pub. Date: Mar. 3, 2022

(54) METHOD OF ESTIMATING POWER DISSIPATED IN FOREIGN OBJECT

(71) Applicant: Medtronic, Inc., Minneapolis, MN (US)

(72) Inventors: David J. Peichel, Minneapolis, MN (US); Eric A. Schilling, Ham Lake, MN (US); Jonathan P. Roberts, Coon Rapids, MN (US); Jacob A. Roe, North St. Paul, MN (US); Joel B. Artmann, Elk River, MN (US); Madeleine HENDERSON, Minneapolis, MN (US)

(21) Appl. No.: 17/008,752

(22) Filed: Sep. 1, 2020

(51) Int. Cl. A61M 1/12; H02J 50/80; H02J 50/60; H02J 50/10

(52) U.S. Cl. CPC A61M 1/127; H02J 50/80; H02J 50/60; A61M 2205/3327; A61M 1/122; A61M 2205/3538; H02J 50/10

(57) ABSTRACT

A method of estimating power dissipated by a foreign metallic object in a transcutaneous energy transfer system (TETS) includes estimating power loss between an external coil of the TETS and an implanted coil of the TETS using a transfer function, the transfer function including inputs, the inputs including: a power supplied to the external coil, a power received by the implanted coil, a measured current within the external coil, and a carrier frequency between the external coil and the implanted coil and generating an alert if the estimated power loss between the external coil and the implanted coil exceeds a predetermined threshold.

### CROSS-REFERENCE TO RELATED APPLICATION

n/a

### FIELD

The present technology is generally related to a system for measuring power dissipated to foreign objects in a transcutaneous energy transfer system (TETS).

### BACKGROUND

Many implantable medical devices have significant energy requirements. A transcutaneous energy transfer system (“TETS”) may be used to power implantable devices including artificial hearts, defibrillators, and electrical systems. Generally, a TETS can transfer energy from an external transmission coil to a receiving coil that is implanted under the skin. A TETS may be used to supplement, replace, or charge an implanted power source such as a rechargeable battery. Using a TETS to power these implantable devices can significantly lessen the potential of infection as the TETS does not require puncturing of the skin and/or wires that pass through the skin. Also, a patient may have increased mobility with the implantable device as power may be transmitted over a range of thicknesses or via an implanted battery.

Proper alignment of the external transmission coil and the implanted receiving coil is critical to transfer energy from the external transmission coil to the receiving coil through an area of the skin, fat, clothing, or air that separates the two coils. If sufficient alignment is not maintained between these two coils, interrupted operation of the implanted medical device may occur. Patient movement may cause the position of the external transmission coil and the receiving coil to shift and not be properly positioned to allow for the desired or required transfer of energy to power the implantable device and/or recharge an implantable battery. Misalignment between the external transmission coil and the receiving coil may further result in undesirable heating of the receiving coil. Moreover, a foreign object proximate the external transmission coil can cause undesirable heating of the foreign object and power losses.

### SUMMARY

The techniques of this disclosure generally relate to methods and systems for detecting power dissipated to a foreign object.

In one aspect, a method of estimating power dissipated by a foreign metallic object in a transcutaneous energy transfer system (TETS) includes estimating power loss between an external coil of the TETS and an implanted coil of the TETS using a transfer function, the transfer function including inputs, the inputs including: a power supplied to the external coil, a power received by the implanted coil, a measured current within the external coil, and a carrier frequency between the external coil and the implanted coil and generating an alert if the estimated power loss between the external coil and the implanted coil exceeds a predetermined threshold.

In another aspect of this embodiment, the alert includes an audible alert indicating a presence of the foreign metallic object.

In another aspect of this embodiment, the alert includes a text alert indicating a presence of the foreign metallic object.

In another aspect of this embodiment, the predetermined threshold is between 0.25 W and 0.5 W.

In another aspect of this embodiment, the method further includes correlating the estimated power loss to the presence of a foreign metal object proximate the external coil.

In another aspect of this embodiment, the TETS includes a controller having an internal battery in communication with the external coil, and wherein the external coil supplies power to the internal battery, and wherein the method further includes reducing power supplied to the internal battery if the estimated power loss between the external coil and the implanted coil exceeds the predetermined threshold.

In another aspect of this embodiment, estimating power loss between an external coil of the TETS and an implanted coil of the TETS using a transfer function occurs over a predetermined period of time.

In another aspect of this embodiment, the method further includes averaging the inputs over the predetermined period time when using the transfer function.

In another aspect of this embodiment, the inputs further include temperature of the external coil and a logarithm of the power outputted by the external coil.

In one aspect, a controller for an implantable blood pump, the implantable blood pump being in communication with transcutaneous energy transfer system (TETS) having an external coil and an implanted coil, includes processing circuitry configured to: estimate power loss between the external coil and the implanted coil using a transfer function, the transfer function including inputs, the inputs including: a power supplied to the external coil, a power received by the implanted coil, a measured current within the external coil, and a carrier frequency between the external coil and the implanted coil and generate an alert if the estimated power loss between the external coil and the implanted coil exceeds a predetermined threshold.

In another aspect of this embodiment, the alert includes an audible alert indicating a presence of the foreign metallic object.

In another aspect of this embodiment, the alert includes a text alert indicating a presence of the foreign metallic object.

In another aspect of this embodiment, the predetermined threshold is between 0.1 W and 1.0 W.

In another aspect of this embodiment, the processing circuitry is further configured to correlate the estimated power loss to the presence of the foreign metallic object proximate the external coil.

In another aspect of this embodiment, the controller includes an internal battery in communication with the external coil, and wherein the external coil supplies power to the internal battery, and wherein the processing circuitry is further configured to reduce power supplied to the internal battery if the estimated power loss between the external coil and the implanted coil exceeds the predetermined threshold.

In another aspect of this embodiment, estimating power loss between an external coil of the TETS and an implanted coil of the TETS using a transfer function occurs over a predetermined period of time.

In another aspect of this embodiment, the processing circuitry is further configured to average the inputs over the predetermined period time when using the transfer function.

In another aspect of this embodiment, the inputs further include temperature of the external coil and a logarithm of the power received by the implanted coil.

In another aspect of this embodiment, the alert is generated following a predetermined period of time.

In one aspect, a method of estimating power dissipated by a foreign metallic object in a transcutaneous energy transfer system (TETS) includes estimating power loss between an external coil of the TETS and an implanted coil of the TETS using a transfer function, the transfer function including inputs, the inputs including: a power supplied to the external coil, a power received by the implanted coil, a measured current within the external coil, a logarithm of the power received by the implanted coil, a temperature of the external coil, and a carrier frequency between the external coil and the implanted coil. The estimated power loss is correlated to the presence of a foreign metal object proximate the external coil. An alert is generated if the estimated power loss between the external coil and the implanted coil exceeds 0.5 W.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an internal system view of an implantable blood pump with a TETS receiver source constructed in accordance with the principles of the present application;

FIG. 2 is an external view of a TETS transmitter and a controller of the system shown in FIG. 1; and

FIG. 3 is a flow chart showing the steps for estimating power dissipated in foreign object.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

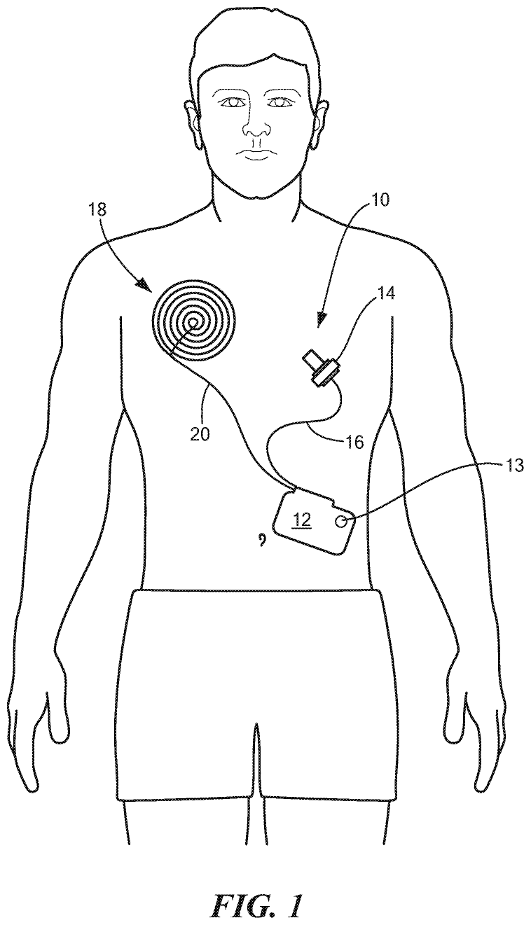

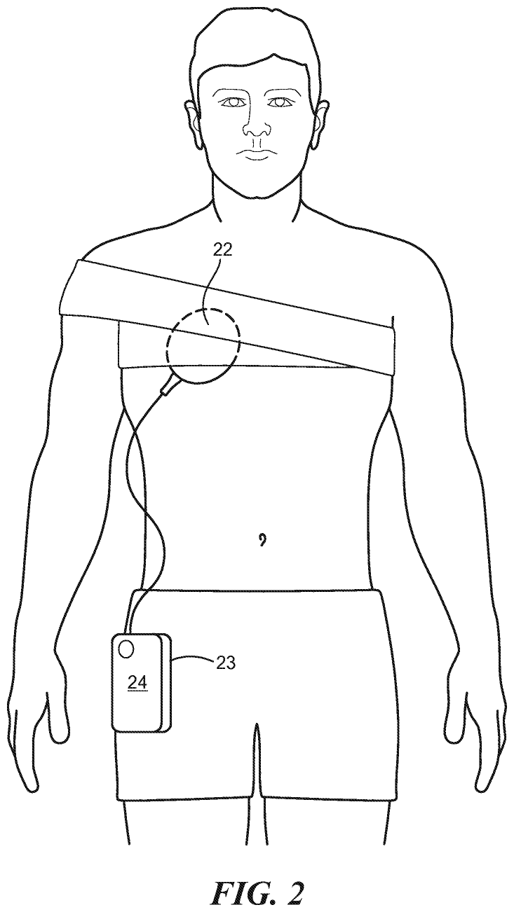

Referring now to the drawings in which like reference designators refer to like elements there is shown in FIGS. 1 and 2 an exemplary mechanical circulatory support device (“MCSD”) constructed in accordance with the principles of the present application and designated generally as “10.” The MCSD 10 may be fully implantable within a patient, whether human or animal, which is to say there are no percutaneous connections between the implanted components of the MCSD 10 and the components outside of the body of the patient. In the configuration shown in FIG. 1, the MCSD 10 includes an internal controller 12 implanted within the body of the patient. The internal controller 12 includes a control circuit having processing circuitry configured to control operation of an implantable blood pump 14. The internal controller 12 may include an internal power source 13, configured to power the components of the controller and provide power to one or more implantable medical devices, for example, the implantable blood pump, such as a ventricular assist device (“VAD”) 14 implanted within the left ventricle of the patient's heart. The power source 13 may include a variety of different types of power sources including an implantable battery. VADs 14 may include centrifugal pumps, axial pumps, or other kinds electromagnetic pumps configured to pump blood from the heart to blood vessels to circulate around the body. One such centrifugal pump is the HVAD and is shown and described in U.S. Pat. No. 7,997,854, the entirety of which is incorporated by reference. One such axial pump is the MVAD and is shown and described in U.S. Pat. No. 8,419,609, the entirety of which is incorporated herein by reference. In an exemplary configuration, the VAD 14 is electrically coupled to the internal controller 12 by one or more implanted conductors 16 configured to provide power to the VAD 14, relay one or more measured feedback signals from the VAD 14, and/or provide operating instructions to the VAD 14.

Continuing to refer to FIG. 1, a receiving or implanted coil 18 may also be coupled to the internal controller 12 by, for example, one or more implanted conductors 20. In an exemplary configuration, the receiving coil 18 may be implanted subcutaneously proximate the thoracic cavity, although any subcutaneous position may be utilized for implanting the receiving coil 18. The receiving coil 18 is configured to be inductively powered through the patient's skin by a transmission or external coil 22 (seen in FIG. 2) disposed opposite the receiving coil 18 on the outside/exterior of the patient's body. For example, as shown in FIG. 2, a transmission coil 22 may be coupled to an external controller 23 having a power source 24, for example, a portable battery carried by the patient or wall power. In one configuration, the battery is configured to generate a radiofrequency signal for transmission of energy from the transmission coil 22 to the receiving coil 18. The receiving coil 18 may be configured for transcutaneous inductive communication with the transmission coil 22 to define a transcutaneous energy transfer system (TETS) that receives power from the transmission coil 22.

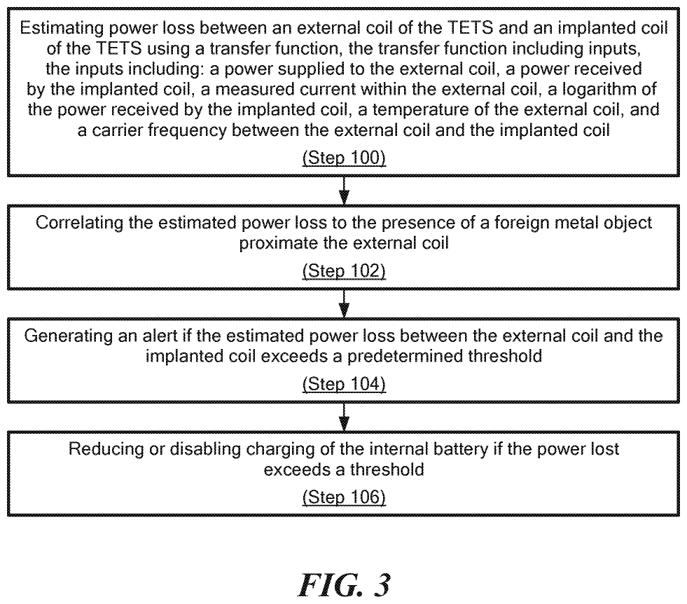

Referring now to FIG. 3, in which a method of estimating power dissipated by a foreign metallic object in a transcutaneous energy transfer system (TETS) is shown. The method includes estimating power loss between an external coil of the TETS and an implanted coil of the TETS using a transfer function (Step 102). The transfer function may include the following inputs: a power supplied to the external coil, a power received by the internal coil, a logarithm of the power received by the internal coil, a measured current within the external coil, and a carrier frequency between the external coil and the implanted coil, voltage supplied to the external coil, a duty cycle of external coil driver, resistance of the external coil and cable; resonance frequency of external and implanted coils, temperature of external coil, differential versus single ended coil drive mode, implanted coil and cable resistance, rectifier output voltage, and configuration of load modulation circuit. Because the carrier frequency between the external coil and the implanted coil is one of the inputs, this may mitigate movement of the external coil with respect to the implanted coil, which may cause a power drop. In other words, use of the carrier frequency factors in power losses associated with movement of the external coil as the patient moves and thus power losses calculated by transfer function may be correlated to the presence of a foreign metallic object (Step 104). Additionally, the inputs may be measured or averaged over a predetermined period time, for example, 5 seconds for inputting into the transfer function.

Moreover, calibration of electrical circuits in manufacturing tests may be used to improve the accuracy of the measured parameters. In particular, the components may be operated during a manufacturing test while measuring the same input parameter with the manufacturing test instrument, and then using the manufacturing test results compared to the device measured value to determine a transfer function or error correction look-up table that will reduce the measurement error of the device measured parameters. Additionally, a system level calibration feature may be included where the power transfer system may be operated when implanted and no foreign metal objects are present. The power transfer system may then be “zeroed out,” in which a calibration coefficient may be determined that would remove any small amount of offset for when no metal objects are present, as well as have the ability to calibrate out any drift in the system over time.

Referring back now to FIG. 3, an alert is generated if the estimated power loss between the external coil and the implanted coil exceeds a predetermined threshold. For example, if the estimated power loss exceeds 0.1 W to 1.0 W, the external controller 23 may generate an audio, tactile, vibratory, or a text alert indicating the presence of a metallic foreign object. In one configuration, the controller 23 may delay generating the alert for a predetermined amount of time, for example, 5 seconds to determine if the metallic foreign object has cleared. For example, small metal objects, such as a coin or a metal pendant carried by the user may cause power to be dissipated to those objects and cause localized heating. However, if the object is removed, is moved, or moves away from the external coil then no alert need to be generated. Thus, the delay in generating the alert prevents providing the user with unneeded alerts for conditions in which the foreign object is only transiently present. In another configuration, power transferred by the external coil 22 to the power source 13 of the internal controller 12 for purposes of charging the power source 13 may be reduced or terminated if power losses estimated by the transfer function exceed the predetermined threshold (Step 106). For example, if power losses exceed 0.25 W to 0.5 W then the power transferred to charge to the internal battery may be temporarily reduced to prevent over heating of the foreign object.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of estimating power dissipated by a foreign metallic object in a transcutaneous energy transfer system (TETS), comprising:
estimating power loss between an external coil of the TETS and an implanted coil of the TETS using a transfer function, the transfer function including inputs, the inputs including: a power supplied to the external coil, a power received by the implanted coil, a measured current within the external coil, and a carrier frequency between the external coil and the implanted coil; and
generating an alert if the estimated power loss between the external coil and the implanted coil exceeds a predetermined threshold.

2. The method of claim 1, wherein the alert includes an audible alert indicating a presence of the foreign metallic object.

3. The method of claim 1, wherein the alert includes a text alert indicating a presence of the foreign metallic object.

4. The method of claim 1, wherein the predetermined threshold is between 0.25 W and 0.5 W.

5. The method of claim 1, further including correlating the estimated power loss to the presence of the foreign metallic object proximate the external coil.

6. The method of claim 1, wherein in the TETS includes a controller having an internal battery in communication with the external coil, and wherein the external coil supplies power to the internal battery, and wherein the method further includes reducing power supplied to the internal battery if the estimated power loss between the external coil and the implanted coil exceeds the predetermined threshold.

7. The method of claim 1, wherein estimating power loss between an external coil of the TETS and an implanted coil of the TETS using a transfer function occurs over a predetermined period of time.

8. The method of claim 7, wherein further including averaging the inputs over the predetermined period time when using the transfer function.

9. The method of claim 1, wherein the inputs further include temperature of the external coil and a logarithm of the power outputted by the external coil.

10. A controller for an implantable blood pump, the implantable blood pump being in communication with transcutaneous energy transfer system (TETS) having an external coil and an implanted coil, the controller comprising:
processing circuitry configured to:
estimate power loss between the external coil and the implanted coil using a transfer function, the transfer function including inputs, the inputs including: a power supplied to the external coil, a power outputted by the external coil, a measured current within the external coil, and a carrier frequency between the external coil and the implanted coil; and
generate an alert if the estimated power loss between the external coil and the implanted coil exceeds a predetermined threshold.

11. The controller of claim 10, wherein the alert includes an audible alert indicating a presence of the foreign metallic object.

12. The controller of claim 10, wherein the alert includes a text alert indicating a presence of the foreign metallic object.

13. The controller of claim 10, wherein the predetermined threshold is between 0.1 W and 1.0 W.

14. The controller of claim 10, wherein the processing circuitry is further configured to correlate the estimated power loss to the presence of the foreign metallic object proximate the external coil.

15. The controller of claim 10, wherein the controller includes an internal battery in communication with the external coil, and wherein the external coil supplies power to the internal battery, and wherein the processing circuitry is further configured to reduce power supplied to the internal battery if the estimated power loss between the external coil and the implanted coil exceeds the predetermined threshold.

16. The controller of claim 10, wherein estimating power loss between an external coil of the TETS and an implanted coil of the TETS using a transfer function occurs over a predetermined period of time.

17. The controller of claim 16, wherein the processing circuitry is further configured to average the inputs over the predetermined period time when using the transfer function.

18. The controller of claim 10, wherein the inputs further include temperature of the external coil and a logarithm of the power received by the implanted coil.

19. The controller of claim 10, wherein the alert is generated following a predetermined period of time.

20. A method of estimating power dissipated by a foreign metallic object in a transcutaneous energy transfer system (TETS), comprising:
estimating power loss between an external coil of the TETS and an implanted coil of the TETS using a transfer function, the transfer function including inputs, the inputs including: a power supplied to the external coil, a power received by the implanted coil, a measured current within the external coil, a logarithm of the power received by the implanted coil, a temperature of the external coil, and a carrier frequency between the external coil and the implanted coil;
correlating the estimated power loss to the presence of a foreign metal object proximate the external coil; and
generating an alert if the estimated power loss between the external coil and the implanted coil exceeds 0.5 W.
